(19) United States
(12) Patent Application Publication
(10) Pub. No.: US 2014/0212987 A1
(43) Pub. Date: Jul. 31, 2014

(54) Signal Ratio in Assay Calibrators

(71) Applicants: Pratap Singh, Wilmington, DE (US); Roland Janzen, Hockessin, DE (US); Jie Li, Middletown, DE (US); Nina Zolotarjova, Wilmington, DE (US)

(72) Inventors: Pratap Singh, Wilmington, DE (US); Roland Janzen, Hockessin, DE (US); Jie Li, Middletown, DE (US); Nina Zolotarjova, Wilmington, DE (US)

(73) Assignee: Siemens Healthcare Diagnostics Inc., Tarrytown, NY (US)

(21) Appl. No.: 13/753,909

(22) Filed: Jan. 30, 2013

(51) Int. Cl. G01N 33/82

(52) U.S. Cl. CPC G01N 33/82
USPC 436/501

(57) ABSTRACT

Methods of enhancing signal ratio between calibrators in an assay for an analyte include conducting an assay for the analyte with zero concentration of analyte in a first calibrator to determine a first signal level. The reagents employed in the assay comprise an antibody reagent comprising an antibody for the analyte wherein a hinge region of the antibody is conjugated to a moiety. The assay for the analyte is also conducted with a second concentration of analyte in a second calibrator to determine a second signal level wherein the second analyte concentration is greater than zero and wherein the reagents employed in the assay comprise the antibody reagent. A ratio of the first signal level to the second signal level is determined and evaluated.

### BACKGROUND

This invention relates to compositions, methods and kits for determining the presence and/or amount of an analyte in a sample suspected of containing the same.

The need to determine many analytes in blood and other biological fluids has become increasingly important in many branches of medicine. The analytes include, among others, therapeutic drugs, drugs of abuse and vitamins, for example.

Typically, immunoassays employ an antibody whose structure recognizes an analyte in a specific manner. The immunoassay is conducted with a signal producing system that produces a detectable change in signal upon binding of the analyte to the antibody. Accordingly, when testing for an analyte in a sample, a detectable change in signal from that produced with a negative sample of a calibrator is taken as a positive result for the presence of that analyte in the sample.

The term “vitamin D” refers to a group of fat-soluble secosteroids. In humans, vitamin D is unique because it can be ingested as cholecalciferol (vitamin D₃) or ergocalciferol (vitamin D₂) and because the body can also synthesize it (from cholesterol) when sun exposure is adequate. Because of this latter property, vitamin D is considered by some to be a non-essential dietary vitamin although most consider it an essential nutrient. Vitamin D has an important physiological role in the positive regulation of calcium ion homeostasis. Vitamin D₃ is the form of the vitamin synthesized by animals. It is also a common supplement added to milk products and certain food products as is vitamin D₂.

Both dietary and intrinsically synthesized vitamin D₃ must undergo metabolic activation to generate bioactive metabolites. In humans, the initial step of vitamin D₃ activation occurs primarily in the liver and involves hydroxylation to form the intermediate metabolite 25-hydroxycholecalciferol (also referred to as calcidiol, calcifediol, 25-hydroxycholecalciferol, or 25-hydroxyvitamin D₃. Calcidiol is the major form of Vitamin D₃ in the circulatory system. Vitamin D₂ also undergoes similar metabolic activation to 25-hydroxyvitamin D₂. Collectively these compounds are called 25-hydroxyvitamin D (abbreviated 25(OH)D) and they are the major metabolites that are measured in serum to determine vitamin D status.

Assessing analyte levels such as, for example, vitamin D levels in biological samples is important since vitamin D deficiency is related to a number of disorders in mammals. There is a need for reagents and methods for accurate and sensitive determinations of concentrations of analytes such as, for example, vitamin D, vitamin D analogs and metabolites thereof in samples.

### SUMMARY

Some examples in accordance with the principles described herein are directed to a method of enhancing signal ratio between calibrators in an assay for an analyte. An assay for the analyte is conducted with zero concentration of analyte in a first calibrator to determine a first signal level. The reagents employed in the assay comprise an antibody reagent comprising an antibody for the analyte wherein a hinge region of the antibody is conjugated to a moiety. The assay for the analyte is also conducted with a second concentration of analyte in a second calibrator to determine a second signal level wherein the second analyte concentration is greater than zero and wherein the reagents employed in the assay comprise the antibody reagent. A ratio of the first signal level to the second signal level is determined and evaluated.

Another example in accordance with the principles described herein is directed to a method of enhancing signal ratio between calibrators in an assay for an analyte. The assay for the analyte is conducted with zero concentration of analyte in a first calibrator to determine a first signal level. Reagents employed in the assay comprise (i) an antibody reagent comprising an antibody having a thioether linkage between a hinge region of the antibody and a small molecule, (ii) a chemiluminescent particle reagent comprising an analyte analog, and (iii) a photosensitizer particle reagent comprising a small molecule-binding moiety. The assay for the analyte is carried out with a second concentration of analyte in a second calibrator to determine a second signal level wherein the second concentration of analyte is greater than zero and wherein the reagents employed in the assay comprise the antibody reagent, the chemiluminescent particle reagent and the photosensitizer particle reagent. A ratio of the first signal level to the second signal level is determined and evaluated.

### DETAILED DESCRIPTION OF SPECIFIC EMBODIMENTS

### General Discussion

Performance of a particular assay format at the low end of a medical decision range can be monitored by monitoring a difference in an amount of signal obtained for calibrators spanning the suspected concentration range of interest of the analyte. A large difference or separation between the signal for calibrators such as, for example, calibrator L1 and calibrator L2 or calibrator L2 and calibrator L3, or calibrator L1 and calibrator L4, for example, is desired. In some examples, five calibrators may be employed, arbitrarily named L1-L5. Signal to noise ratio may be evaluated by determining an amount of signal using a calibrator that contains no analyte, arbitrarily designated calibrator L1 (background), and the amount of signal obtained for a calibrator containing a first known amount of analyte above zero, arbitrarily designated calibrator L2. This evaluation may also include determining an amount of signal using calibrator L1 and the amount of signal for a calibrator containing a second known amount of analyte above zero, arbitrarily designated L3. Such an evaluation would also include such determination using calibrators L4 and L5, wherein each of the calibrators contains known increasing amounts of analyte. Depending on the assay format, the difference in signal may be an increase in signal or a decrease in signal. For example, for a competitive assay, in most instances there will be a decrease in signal related to the concentration of analyte; and, for a sandwich assay, in most instances there will be an increase in signal related to the concentration of analyte.

Examples in accordance with the principles described herein provide for better performance in an assay for an analyte compared to reagents not in accordance with the principles described herein. In some examples in accordance with the principles described herein, the difference between the signal or ratio of signals for calibrators, for example, L1 and L5 (where the ratio is L1/L5 for assays that produce a decrease in signal as the concentration of analyte increases or L5/L1 for assay that produce an increase in signal as the concentration of analyte increases), using as one reagent in an assay, an antibody reagent comprising an antibody for the analyte wherein a hinge region of the antibody is conjugated to a moiety is at least 4 times greater, or at least 5 times greater, or at least about 6 times greater, or at least about 7 times greater, or at least about 8 times greater, compared to the corresponding assay employing an antibody reagent wherein the antibody is conjugated to the same moiety at a region other than the hinge region. The signal ratio between calibrators is the ratio of signal or signal level obtained for one calibrator to signal or signal level obtained for a second calibrator where the concentration of analyte in the calibrators is different.

In some instances, the results of the assays using the calibrators are also presented in a graph format wherein the amount of signal is plotted against the concentration of the calibrators. In accordance with examples using assay reagents that include an antibody reagent in accordance with the principles described herein, the slope of the line between calibrator L1 and calibrator L2 generally is steeper compared with results obtained with assay reagents that include an antibody reagent that is not in accordance with the principles described herein. Furthermore, the slope of the line from calibrator L1 to calibrator L5 is usually steeper with examples using assay reagents that include an antibody reagent in accordance with the principles described herein compared with results obtained with assay reagents that are not in accordance with the principles described herein.

### Antibody Reagent

As mentioned above, the reagents employed in the assay for determining a set of calibrators comprise an antibody reagent comprising an antibody for the analyte wherein a hinge region of the antibody is conjugated to a moiety. In some examples in accordance with the principles described herein, the antibody is one that is capable of generating more cysteine residues in the hinge region of the antibody when compared to the number of cysteine residues generated in the hinge region of another antibody. Antibodies include various classes and isotypes, such as IgA, IgD, IgE, IgG1, IgG2a, IgG2b and IgG3, and IgM, for example. Fragments thereof may include Fab, Fv and F(ab′)₂, and Fab′, for example. In addition, aggregates, polymers, and conjugates of immunoglobulins or their fragments can be used where appropriate so long as binding affinity for analyte is retained. Antibodies for an analyte may be prepared by techniques including, but not limited to, immunization of a host and collection of sera (polyclonal), preparing continuous hybrid cell lines and collecting the secreted protein (monoclonal) or cloning and expressing nucleotide sequences or mutagenized versions thereof coding at least for the amino acid sequences required for specific binding of natural antibodies, for example. The source of the antibody (polyclonal or monoclonal) may be any source that is capable of producing an antibody in response to immunization with an immunogen of interest. The source may be an animal such as, but not limited to, a mouse, a sheep, a goat, a rabbit, a rat, or a human, for example. In one example in accordance with the principles described herein, the antibody is a monoclonal or polyclonal antibody from a sheep source. Applicant has found that antibodies from a sheep source are capable of generating more cysteine residues in the hinge region of the antibody when compared to the number of cysteine residues generated in the hinge region of an antibody from another source.

The moiety that is conjugated to an antibody may be a member of a specific binding pair (sbp member) or a member of a signal producing system, for example.

The sbp member is one of two different molecules, having an area on the surface or in a cavity, which specifically binds to and is thereby defined as complementary with a particular spatial and polar organization of the other molecule. Specific binding involves the specific recognition of one of two different molecules for the other compared to substantially less recognition of other molecules. On the other hand, non-specific binding involves non covalent binding between molecules that is relatively independent of specific surface structures. Non-specific binding may result from several factors including hydrophobic interactions between molecules. The term “sbp members” includes, but is not limited to, small molecules and binding partners for small molecules, which may be, for example, members of an immunological pair such as antigen and antibody, biotin and avidin or streptavidin, fluorescein and antibody for fluorescein, rhodamine and antibody for rhodamine, a benzene derivative (such as, for example, nitrobenzene, dinitrobenzene and tri-nitrobenzene and corresponding carboxylic acids) and antibody for the benzene derivative, hormones and hormone receptors, enzyme substrate and enzyme. Other sbp members include nucleic acid duplexes, IgG protein A, and polynucleotide pairs such as DNA DNA, DNA RNA, for example.

As mentioned above, in some examples in accordance with the principles described herein, the moiety may be a member of a signal producing system. The signal producing system may have one or more components, at least one component being a label. The signal producing system generates a signal that relates to the presence of vitamin D in a sample. The signal producing system includes all of the reagents required to produce a measurable signal. Other components of the signal producing system may be included in a developer solution and can include, but are not limited to, substrates, enhancers, activators, chemiluminescent compounds, cofactors, inhibitors, scavengers, metal ions, and specific binding substances required for binding of signal generating substances, for example. Other components of the signal producing system may be coenzymes, substances that react with enzymic products, other enzymes and catalysts, for example. The signal producing system provides a signal detectable by external means, by use of electromagnetic radiation, desirably by visual examination. Exemplary signal-producing systems are described in U.S. Pat. No. 5,508,178, the relevant disclosure of which is incorporated herein by reference.

The term “label” includes poly(amino acid) labels and non-poly(amino acid) labels. The term “poly(amino acid) label moieties” includes labels that are proteins such as, but not limited to, enzymes, antibodies, peptides, and immunogens, for example. With label proteins such as, for example, enzymes, the molecular weight range will be from about 10,000 to about 600,000, or from about 10,000 to about 300,000 molecular weight. There is usually at least one compound in accordance with the principles described herein (analog group) per about 200,000 molecular weight, or at least about 1 per about 150,000 molecular weight, or at least about 1 per about 100,000 molecular weight, or at least about 1 per about 50,000 molecular weight, for example, of the protein. In the case of enzymes, the number of analog groups is usually from 1 to about 20, about 2 to about 15, about 3 to about 12, or about 6 to about 10.

Enzymes include, by way of illustration and not limitation, redox enzymes such as, for example, dehydrogenases, e.g., glucose-6-phosphate dehydrogenase and lactate dehydrogenase; enzymes that involve the production of hydrogen peroxide and the use of the hydrogen peroxide to oxidize a dye precursor to a dye such as, for example, horseradish peroxidase, lactoperoxidase and microperoxidase; hydrolases such as, for example, alkaline phosphatase and β-galactosidase; luciferases such as, for example firefly luciferase, and bacterial luciferase; transferases; combinations of enzymes such as, but not limited to, saccharide oxidases, e.g., glucose and galactose oxidase, or heterocyclic oxidases, such as uricase and xanthine oxidase, coupled with an enzyme that employs hydrogen peroxide to oxidize a dye precursor, that is, a peroxidase such as horseradish peroxidase, lactoperoxidase or microperoxidase, for example.

The term “non-poly(amino acid) labels” includes those labels that are not proteins. The non-poly(amino acid) label is capable of being detected directly or is detectable through a reaction that produces a detectable signal. The non-poly(amino acid) label can be isotopic or non-isotopic and can be, by way of illustration and not limitation, a radioisotope, a luminescent compound (which includes, but is not limited to acridinium esters, fluorescent compounds and chemiluminescent compounds, for example), a polynucleotide coding for a catalyst, a promoter, a dye, a coenzyme, an enzyme substrate, a radioactive group, and an amplifiable polynucleotide sequence, for example. In some example, the non-poly(amino acid) labels are radioisotopic, luminescent (such as, e.g., acridinium esters), particulate (such as, e.g., magnetic particles that can be separated bound from un-bound, latex particles that can be measured by turbidity and nephelometry, and chemiluminescence beads (e.g., LOCI chemibeads), for example.

Magnetic particles include paramagnetic particles, ferromagnetic particles and diamagnetic particles. Such particles include, but are not limited to, transition metals of periods 4-7 of the Periodic Table including chromium, copper, cobalt, aluminum, manganese, iron, and nickel, for example.

Chemiluminescent particles are particles that have associated therewith a chemiluminescent compound. The phrase “associated therewith” as used herein means that a compound such as, for example, a chemiluminescent compound and a particle may be associated by direct or indirect bonding, adsorption, absorption, incorporation, or solution, for example. Examples of chemiluminescent compounds that may be utilized are those set forth in U.S. Pat. Nos. 5,340,716 and 6,251,581, the relevant disclosures of which are incorporated herein by reference. In some examples, the chemiluminescent compound is a photoactivatable substance that undergoes a chemical reaction upon direct or sensitized excitation by light or upon reaction with singlet oxygen to form a metastable reaction product that is capable of decomposition with the simultaneous or subsequent emission of light, usually within the wavelength range of 250 to 1200 nm. The term “photoactivatable” includes “photochemically activatable”. In some examples, the chemiluminescent compounds are those that react with singlet oxygen to form dioxetanes or dioxetanones. The latter are usually electron rich olefins. Exemplary of such electron rich olefins are enol ethers, enamines, 9-alkylidene-N-alkylacridans, arylvinylethers, dioxenes, arylimidazoles, 9-alkylidene-xanthanes and lucigenin Other compounds include luminol and other phthalhydrazides and chemiluminescent compounds that are protected from undergoing a chemiluminescent reaction by virtue of their being protected by a photochemically labile protecting group, such compounds including, for example, firefly luciferin, aquaphorin, and luminol. Examples of such chemiluminescent compounds that may be utilized are those set forth in U.S. Pat. No. 5,709,994, the relevant disclosure of which is incorporated herein by reference.

Sensitizer particles are particles that have associated therewith a sensitizer compound, which includes, but is not limited to, a photosensitizer compound. Examples of sensitizer compounds that may be utilized are those set forth in U.S. Pat. Nos. 5,340,716 and 6,251,581, the relevant disclosures of which are incorporated herein by reference.

A photosensitizer is a sensitizer for generation of singlet oxygen usually by excitation with light. In some examples, the photosensitizer absorbs at a longer wavelength than the chemiluminescent compound and has a lower energy triplet than the chemiluminescent compound. The photosensitizer can be photoactivatable (e.g., dyes and aromatic compounds). The photosensitizer is usually a compound comprised of covalently bonded atoms, usually with multiple conjugated double or triple bonds. The compound should absorb light in the wavelength range of 200-1100 nm, usually 300-1000 nm, preferably 450-950 nm. Typical photosensitizers include, but are not limited to, acetone, benzophenone, 9-thioxanthone, eosin, 9,10-dibromoanthracene, methylene blue, metallo-porphyrins (e.g., hematoporphyrin), phthalocyanines, chlorophylls, rose bengal, buckminsterfullerene, for example, and derivatives of these compounds. Examples of other photosensitizers are enumerated in N. J. Turro, “Molecular Photochemistry”, page 132, W. A. Benjamin Inc., N.Y. 1965. The photosensitizer assists photoactivation where activation is by singlet oxygen. Usually, the photosensitizer absorbs light and the thus formed excited photosensitizer activates oxygen to produce singlet oxygen, which reacts with the chemiluminescent compound to give a metastable luminescent intermediate.

### Preparation of Antibody Reagent

In some examples in accordance with the principles described herein, the conjugate of the moiety and an antibody wherein the moiety is linked to the hinge region of the antibody may be prepared by introducing free sulfhydryl groups in the hinge region of the antibody. The hinge region comprises free cysteine groups or disulfide groups formed from cysteine groups; disulfides are known to be present on the heavy chain of an antibody. The preparation of the conjugate in accordance with the principles described herein involves reduction of the disulfide groups to free sulfhydryl or thiol groups. The reduction may be achieved by treating the antibody with a reducing agent under conditions for achieving reduction of disulfide bonds. Suitable reducing agents include, but are not limited to, sulfur-containing reducing agents such as, for example, dithiothreitol (DTT), and 2-mercaptoethanol (2ME), dithioerythritol (DTE), cysteine, mercaptoacetic acid, 2-aminoethanethiol, N-acetyl cysteine, for example, or other reducing agents such as, for example, a borohydride, e.g., sodium borohydride or pyridine borane, or a phosphine, e.g., tris-(2-carboxyethyl) phosphine hydrochloride, and bisulfite solutions especially a metabisulfite solution (MBS) or sodium bisulfite, and including combinations of two or more of the above reducing agents that are compatible with one another. The reaction conditions are dependent on the nature of the antibody, the nature of the reducing agent, and the extent of reduction desired to incorporate (generate) the number of free thiol groups in the reduced protein, for example. The reaction conditions are those that are sufficient for achieving reduction of all or a limited number of sulfide groups to free thiol groups. In some examples, the reaction may be carried out at a temperature of about 2° C. to about 50° C., or about 2° C. to about 40° C., or about 2° C. to about 30° C., or about 2° C. to about 20° C., or about 2° C. to about 10° C., or about 10° C. to about 40° C., or about 10° C. to about 30° C., or about 10° C. to about 20° C., about 20° C. to about 50° C., or about 20° C. to about 40° C., or about 20° C. to about 35° C., or about 25° C. to about 50° C., or about 25° C. to about 40° C., or about 25° C. to about 35° C., or about 30° C. to about 50° C., or about 30° C. to about 40° C., for example. In some examples, the pH of the reaction is about 5 to about 8, or about 5 to about 7, or about 5 to about 6, or about 6 to about 8, or about 6 to about 7, for example. In some examples, the reaction is carried out for about 0.5 hours to about 16 hours, or about 0.5 hours to about 10 hours, or about 0.5 hours to about 6 hours, or about 0.5 hours to about 5 hours, or about 0.5 hours to about 4 hours, or about 0.5 hours to about 3 hours, or about 0.5 hours to about 2 hours, or about 0.5 hours to about 1 hour, for example. After the reduction reaction, the resulting antibody may be purified by filtration such as, e.g., diafiltration; chromatography such as, e.g., size exclusion chromatography; dialysis, or a combination of two or more of the above.

The reduced antibody is then reacted with a desired moiety, which is linked either directly or through a linking group to a sulfhydryl-reactive entity such as, by way of illustration and not limitation, a haloacetyl (bromoacetyl, iodoacetyl, chloroacetyl) group, a maleimide, or an epoxy group, for example. The reaction conditions are dependent on the nature of the antibody, the nature of the sulfhydryl-reactive entity, the nature of the linking group, or the extent to which the desired moiety is incorporated into the antibody, for example. The reaction conditions are those that are sufficient to achieve linking of the moiety to the sulfhydryl groups of the antibody. In some examples, the reaction may be carried out at a temperature of about 2° C. to about 50° C., or about 2° C. to about 40° C., or about 2° C. to about 30° C., or about 2° C. to about 20° C., or about 2° C. to about 10° C., or about 10° C. to about 40° C., or about 10° C. to about 30° C., or about 10° C. to about 20° C., about 20° C. to about 50° C., or about 20° C. to about 40° C., or about 20° C. to about 35° C., or about 20° C. to about 30° C., or about 20° C. to about 25° C., for example. In some examples, the pH of the reaction is about 5 to about 8, or about 5 to about 7, or about 5 to about 6, or about 6 to about 8, or about 6 to about 7, for example. In some examples, the reaction is carried out for about 0.5 hours to about 16 hours, or about 0.5 hours to about 10 hours, or about 1 to about 6 hours, or about 1 to about 5 hours, or about 1 to about 4 hours, or about 1 to about 3 hours, or about 2 to about 5 hours, or about 2 to about 4 hours, for example. After the reaction, the resulting antibody-moiety conjugate may be purified by filtration such as, e.g., diafiltration; chromatography such as, e.g., size exclusion chromatography; dialysis, or a combination of two or more of the above. In some examples, the reaction product is treated to quench unreacted sulfhydryl groups. Quenching agents include, but are not limited to N-ethylmaleimide, iodoacetic acid, or iodoacetamide, for example.

The linking group may be selected to achieve certain desirable characteristics such as increased aqueous solubility, desirable sensitivity to light, heat or reactivity with the antibody. The linking group employed may be a chain of from 1 to about 30 or more atoms, from about 1 to about 20 atoms, about 1 to about 10 atoms, each independently selected from the group normally consisting of carbon, oxygen, sulfur, nitrogen, and phosphorous, usually carbon and oxygen. The number of heteroatoms in the linking group normally ranges from about 0 to about 8, from about 1 to about 6, about 2 to about 4. The number of atoms in the chain is determined by counting the number of atoms other than hydrogen or other monovalent atoms along the shortest route between the substructures being connected. The atoms of the linking group may be substituted with atoms other than hydrogen such as carbon and oxygen, for example, in the form, e.g., of alkyl, aryl, aralkyl, hydroxyl, alkoxy, aryloxy, and aralkoxy, for example. As a general rule, the length of a particular linking group can be selected arbitrarily to provide for convenience of synthesis with the proviso that there be minimal interference caused by the linking group with the ability of the linked molecules to perform their function related to an assay in question.

The linking group may be aliphatic or aromatic. When heteroatoms are present, oxygen will normally be present as oxy or oxo, bonded to carbon, sulfur, nitrogen or phosphorous; sulfur will be present as thioether or thiono; nitrogen will normally be present as nitro, nitroso or amino, normally bonded to carbon, oxygen, sulfur or phosphorous; phosphorous will be bonded to carbon, sulfur, oxygen or nitrogen, usually as phosphonate and phosphate mono- or diester. Functionalities present in the linking group may include esters, thioesters, amides, thioamides, ethers including polyethers such as, for example, polyethylene glycols, ureas, thioureas, guanidines, azo groups, thioethers, and carboxylate, for example.

### General Description of Assays for an Analyte

An antibody reagent in accordance with the principles described herein may be employed in any assay for an analyte that employs an antibody reagent wherein an antibody is conjugated to a moiety. Such assays are referred to as immunoassays. The assays may be conducted on the sample as an immediate continuation of the treatment of the sample with a releasing agent, if necessary, to release analyte from endogenous binding substances, or the assay may be carried out at a point thereafter. The assays are conducted by combining in an assay medium the sample with reagents for determining the amount of the analyte in the sample at least one of which is an antibody reagent in accordance with the principles described herein. The nature of the reagents is dependent on the particular type of assay to be performed. The combination in the medium is subjected to conditions for binding of the analyte to the antibody of the antibody reagent to form a complex. The amount of the complex is measured where the amount of the complex is related to one or both of the presence and amount of analyte in the sample.

The analyte that is used to form the calibrators is, in most instances, one that is present or suspected of being present in a sample to be analyzed in the assay for the analyte, for which the calibrators are employed. The analyte may be synthetic or non-synthetic, that is, one that is extracted from the sample or the sample itself may be employed as long as the concentration of the analyte in the various calibrators is known. The sample may be a solid, semi-solid or a fluid (a liquid or a gas) from any source. The sample may be biological samples or non-biological samples. The phrase “biological sample” refers to any biological material such as, for example, body fluid, body tissue, body compounds and culture media. In some examples the sample may be a body excretion, a body aspirant, a body excisant or a body extractant. The body may be mammalian or non-mammalian. Mammalian subjects may be, e.g., humans or other animal species. In some examples, the body is a human body. Body excretions are those substances that are excreted from a body (although they also may be obtained by excision or extraction) such as, for example, urine, feces, stool, vaginal mucus, semen, tears, cerebral spinal fluid, lymphatic fluid, breath, sweat, blister fluid and inflammatory exudates. Body excisants are those materials that are excised from a body such as, for example, skin, hair and tissue samples including biopsies from organs and other body parts. Body aspirants are those materials that are aspirated from a body such as, for example, mucus, saliva and sputum. Body extractants are those materials that are extracted from a body such as, for example, whole blood, plasma, serum, spinal fluid, cerebral spinal fluid, lymphatic fluid, synovial fluid and peritoneal fluid. In many instances, the biological sample is whole blood, plasma or serum. “Non-biological samples” are those that do not relate to a biological material and include, but are not limited to, soil samples, water samples, air samples, samples of other gases and mineral samples and waste streams, for example. In some examples the analyte is vitamin D and the antibody reagent in accordance with the principles described herein comprises an antibody for vitamin D.

For conducting an assay for an analyte, the analyte or the sample can be prepared in an assay medium, which is discussed more fully hereinbelow. In some instances a pretreatment may be applied to the sample such as, for example, to lyse blood cells. In some examples, such pretreatment is performed in a medium that does not interfere subsequently with an assay.

The assay employed can be performed either without separation (homogeneous) or with separation (heterogeneous) of any of the assay components or products. The assay may be competitive or non-competitive and involve labeled or non-labeled reagents. Heterogeneous assays usually involve one or more separation steps. Immunoassays involving non-labeled reagents usually comprise the formation of relatively large complexes involving one or more antibodies prepared from immunogenic conjugates in accordance with the principles described herein. Such assays include, for example, immunoprecipitin and agglutination methods and corresponding light scattering techniques such as, e.g., nephelometry and turbidimetry, for the detection of antibody complexes. Labeled immunoassays include, but are not limited to, chemiluminescence immunoassays, enzyme immunoassays, fluorescence polarization immunoassays, radioimmunoassays, radial partition immunoassays, inhibition assays, induced luminescence assays, and fluorescent oxygen channeling assays, for example.

One general group of immunoassays includes immunoassays using a limited concentration of an antibody reagent in accordance with the principles described herein. Another group of immunoassays involves the use of an excess of one or more of the principal reagents such as, for example, an excess of an antibody reagent in accordance with the principles described herein. Another group of immunoassays includes separation-free homogeneous assays in which a labeled antibody reagent in accordance with the principles described herein modulates the label signal upon binding of an antibody reagent in accordance with the principles described herein to an analyte or analyte analog.

As mentioned above, the assays can be performed either without separation (homogeneous) or with separation (heterogeneous) of any of the assay components or products. Homogeneous immunoassays are exemplified by the EMIT® assay (Siemens Healthcare Diagnostics Inc., Deerfield, Ill.) disclosed in Rubenstein, et al., U.S. Pat. No. 3,817,837, column 3, line 6 to column 6, line 64; immunofluorescence methods such as those disclosed in Ullman, et al., U.S. Pat. No. 3,996,345, column 17, line 59, to column 23, line 25; enzyme channeling immunoassays (“ECIA”) such as those disclosed in Maggio, et al., U.S. Pat. No. 4,233,402, column 6, line 25 to column 9, line 63; the fluorescence polarization immunoassay (“FPIA”) as disclosed, for example, in, among others, U.S. Pat. No. 5,354,693; and some enzyme immunoassays. Exemplary of heterogeneous assays are the radioimmunoassay, disclosed in Yalow, et al., J. Clin. Invest. 39:1157 (1960) and the enzyme linked immunosorbant assay (“ELISA”). The relevant portions of the above disclosures are all incorporated herein by reference.

Other enzyme immunoassays are the enzyme modulate mediated immunoassay (“EMMIA”) discussed by Ngo and Lenhoff, FEBS Lett. (1980) 116:285-288; the substrate labeled fluorescence immunoassay (“SLFIA”) disclosed by Oellerich, J. Clin. Chem. Clin. Biochem. (1984) 22:895-904; the combined enzyme donor immunoassays (“CEDIA”) disclosed by Khanna, et al., Clin. Chem. Acta (1989) 185:231-240; homogeneous particle labeled immunoassays such as particle enhanced turbidimetric inhibition immunoassays (“PETINIA”), particle enhanced turbidimetric immunoassay (“PETIA”); the Affinity Chromium dioxide Mediated Immuno Assay (“ACMIA”) assay format, which is described in U.S. Pat. Nos. 7,186,518, 5,147,529, 5,128,103, 5,158,871, 4,661,408, 5,151,348, 5,302,532, 5,422,284, 5,447,870, and 5,434,051, the disclosures of which are incorporated herein in their entirety; for example.

Other assays include acridinium ester label assays such as those discussed in U.S. Pat. Nos. 6,355,803; 6,673,560; 7,097,995 and 7,319,041, the relevant disclosures of which are incorporated herein by reference. A particular example of an acridinium ester label assay is an acridinium ester label immunoassay using paramagnetic particles as a solid phase (“ADVIA” immunoassay). Other assays include the sol particle immunoassay (“SPIA”), the disperse dye immunoassay (“DIA”); the metalloimmunoassay (“MIA”); the enzyme membrane immunoassays (“EMIA”); and luminoimmunoassays (“LIA”). Other types of assays include immunosensor assays involving the monitoring of the changes in the optical, acoustic and electrical properties of the present conjugate upon the binding of analyte. Such assays include, for example, optical immunosensor assays, acoustic immunosensor assays, semiconductor immunosensor assays, electrochemical transducer immunosensor assays, potentiometric immunosensor assays, amperometric electrode assays.

Heterogeneous assays usually involve one or more separation steps and can be competitive or non-competitive. A variety of competitive and non-competitive heterogeneous assay formats are disclosed in Davalian, et al., U.S. Pat. No. 5,089,390, column 14, line 25 to column 15, line 9, incorporated herein by reference. In an example of a competitive heterogeneous assay, a support having an antibody for analyte bound thereto is contacted with a medium containing the sample suspected of containing the analyte and a an analyte analog that comprises a label. The antibody is bound to the support by virtue of comprising a binding partner for a small molecule, which binds to a small molecule attached to the hinge region of the antibody in accordance with the principles described herein. Analyte in the sample competes, for binding to the analyte antibody, with the labeled analyte analog. After separating the support and the medium, the label activity of the support or the medium is determined by conventional techniques and is related to the amount of analyte in the sample. In a variation of the above competitive heterogeneous assay, the support comprises an analyte analog, which competes with analyte of the sample for binding to an antibody reagent in accordance with the principles described herein.

In some examples in accordance with the principles described herein, the sample to be analyzed is subjected to a pretreatment to release analyte from endogenous binding substances such as, for example, plasma or serum proteins that bind the analyte. The release of the analyte from endogenous binding substances may be carried out, for example, by addition of a digestion agent or a releasing agent or a combination of a digestion agent and a releasing agent used sequentially. The digestion agent is one that breaks down the endogenous binding substances so that they can no longer bind the analyte. Such agents include, but are not limited to, proteinase K and proteinase K and protein denaturing agents such as, e.g., detergents (sodium dodecyl sulfate, for example). Releasing agents for releasing the analyte from endogenous binding substances include, by way of illustration and not limitation, acidic denaturing agents such as, for example, salicylic acid, warfarin, sulfonic acids, toluene sulfonic acids, naphthalene sulfonic acid, anilinonaphthalene sulfonic acids (ANS) (including, e.g., 1-anilinonaphthalene-8-sulfonic acid (1,8-ANS) and 8-anilinonapthalene-1-sulfonic acid (8-ANS)), salicylic acids and derivatives of the above, and chaotropic agents including, but not limited to, sodium trichloroacetate, for example.

The conditions such as, for example, duration, temperature, pH and concentration of the releasing agent in the medium for carrying out the digestion or releasing actions are dependent on the nature of the analyte, the nature of the endogenous binding substances, the nature of the sample, and the nature of the releasing agent, for example. In general, the conditions are sufficient to achieve the desired effect or function. In some examples in accordance with the principles described herein, an effective concentration of releasing agent is about 0.01 to about 200 mg/mL, or about 0.01 to about 150 mg/mL, or about 0.01 to about 100 mg/mL, or about 0.01 to about 80 mg/mL, or about 0.01 to about 60 mg/mL, or about 0.01 to about 40 mg/mL, or about 0.01 to about 20 mg/mL, or about 0.01 to about 10 mg/mL, or about 0.01 to about 5 mg/mL, or about 0.1 to about 200 mg/mL, or about 0.1 to about 100 mg/mL, or about 0.1 to about 20 mg/mL, or about 0.1 to about 10 mg/mL, or about 0.1 to about 5 mg/mL, or about 0.1 to about 1 mg/mL. The pretreatment of the sample to release the analyte from endogenous binding substances may be carried out as a separate step prior to conducting an assay or as a first step in an assay. In either case, one or more reagents may be required to stop the action of the digestion agent and/or the releasing agent.

The conditions for conducting the assays include carrying out the assay in an aqueous buffered medium at a moderate pH, generally that which provides optimum assay sensitivity. The aqueous medium may be solely water or may include from 0.1 to about 40 volume percent of a cosolvent. The pH for the medium will be in the range of about 4 to about 11, or in the range of about 5 to about 10, or in the range of about 6.5 to about 9.5, for example. The pH will usually be a compromise between optimum binding of the binding members of any specific binding pairs, the pH optimum for other reagents of the assay such as members of the signal producing system, and so forth. Various buffers may be used to achieve the desired pH and maintain the pH during the assay. Illustrative buffers include, by way of illustration and not limitation, borate, phosphate, carbonate, TRIS, barbital, PIPES, HEPES, MES, ACES, MOPS, and BICINE, for example. The particular buffer employed is not critical, but in an individual assay one or another buffer may be preferred.

Various ancillary materials may be employed in the assay methods. For example, in addition to buffers the medium may comprise stabilizers for the medium and for the reagents employed. In some embodiments, in addition to these additives, proteins may be included, such as, for example, albumins; organic solvents such as, for example, formamide; quaternary ammonium salts; polyanions such as, for example, dextran sulfate; binding enhancers, for example, polyalkylene glycols; polysaccharides such as, for example, dextran or trehalose. The medium may also comprise agents for preventing the formation of blood clots. Such agents are well known in the art and include, but are not limited to, EDTA, EGTA, citrate, heparin, for example. The medium may also comprise one or more preservatives such as, but not limited to, sodium azide, neomycin sulfate, PROCLIN® 300, Streptomycin, for example. The medium may additionally comprise one or more surfactants. Any of the above materials, if employed, is present in a concentration or amount sufficient to achieve the desired effect or function.

One or more incubation periods may be applied to the medium at one or more intervals including any intervals between additions of various reagents employed in an assay including those mentioned above. The medium is usually incubated at a temperature and for a time sufficient for binding of various components of the reagents and binding of the analyte in the sample to occur. Moderate temperatures are normally employed for carrying out the method and usually constant temperature, preferably, room temperature, during the period of the measurement. In some examples, incubation temperatures range from about 5° to about 99° C., or from about 15° C. to about 70° C., or from about 20° C. to about 45° C., for example. The time period for the incubation, in some examples, is about 0.2 seconds to about 24 hours, or about 1 second to about 6 hours, or about 2 seconds to about 1 hour, or about 1 minute to about 15 minutes, for example. The time period depends on the temperature of the medium and the rate of binding of the various reagents, which is determined by the association rate constant, the concentration, the binding constant and dissociation rate constant.

Some known assays utilize a signal producing system (sps) that employs first and second sps members. The designation “first” and “second” is completely arbitrary and is not meant to suggest any order or ranking among the sps members or any order of addition of the sps members in the present methods. The sps members may be related in that activation of one member of the sps produces a product such as, for example, light or an activated product, which results in activation of another member of the sps.

In some embodiments of assays, the sps members comprise a sensitizer such as, for example, a photosensitizer, and a chemiluminescent composition where activation of the sensitizer results in a product that activates the chemiluminescent composition. The second sps member usually generates a detectable signal that relates to the amount of bound and/or unbound sps member, i.e., the amount of sps member bound or not bound to the vitamin D analyte being detected or to a compound in accordance with the principles described herein. In some examples in accordance with the principles described herein, one of either the sensitizer reagent or the chemiluminescent reagent comprises an antibody reagent in accordance with the principles described herein.

As mentioned above, an example in accordance with the principles described herein is directed to a method of enhancing signal ratio between calibrators in an assay for an analyte. The assay for the analyte is conducted with zero concentration of analyte in a first calibrator to determine a first signal level. Reagents employed in the assay comprise (i) an antibody reagent in accordance with the principles described herein, (ii) a chemiluminescent particle reagent comprising an analyte analog, and (iii) a photosensitizer particle reagent comprising a small molecule-binding moiety or a binding partner for the small molecule. The assay for the analyte is carried out with a second concentration of analyte in a second calibrator to determine a second signal level wherein the second concentration of analyte is greater than zero and wherein the reagents employed in the assay comprise the antibody reagent, the chemiluminescent particle reagent and the photosensitizer particle reagent. A ratio of the first signal level to the second signal level is determined and evaluated.

In a particular example, an induced luminescence immunoassay may be employed. The induced luminescence immunoassay is referred to in U.S. Pat. No. 5,340,716 (Ullman), which disclosure is incorporated herein by reference. In one approach, the assay uses a particle having associated therewith a photosensitizer where a vitamin D analog is bound to the particle (particle-analog reagent). The chemiluminescent reagent comprises an antibody for vitamin D, which is an antibody in accordance with the principles described herein wherein antibody is linked through the hinge region to a small molecule that is in turn bound to a binding partner for the small molecule on a chemiluminescent particle. The vitamin D analyte competes with the particle-analog reagent for binding to the antibody for vitamin D. If the vitamin D analyte is present, the fewer is the number of molecules of particle-analog reagent that come into close proximity with the chemiluminescent reagent. Therefore, there will be a decrease in the assay signal. The photosensitizer generates singlet oxygen and activates the chemiluminescent reagent when the two labels are in close proximity. The activated chemiluminescent reagent subsequently produces light. The amount of light produced is related to the amount of the complex formed, which in turn is related to the amount of vitamin D analyte present in a calibrator or a sample.

In another particular example of an induced luminescence immunoassay, the assay uses a particle having associated therewith a chemiluminescent compound where a vitamin D analog is bound to the particle (particle-analog reagent). The photosensitizer reagent comprises an antibody for vitamin D, which is in accordance with the principles described herein wherein antibody is linked through the hinge region to a small molecule that is in turn bound to a binding partner for the small molecule on a chemiluminescent particle. The vitamin D analyte competes with the particle-analog reagent for binding to the antibody for vitamin D. If the vitamin D analyte is present, the fewer is the number of molecules of particle-analog reagent that come into close proximity with the photosensitizer reagent. Therefore, there will be a decrease in the assay signal. The photosensitizer generates singlet oxygen and activates the chemiluminescent compound of the particle-analog reagent when the two labels are in close proximity. The activated chemiluminescent compound subsequently produces light. The amount of light produced is related to the amount of the complex formed, which in turn is related to the amount of vitamin D analyte present in a calibrator or a sample.

In a particular example of an induced luminescence assay, a photosensitizer particle is employed that is conjugated to a binding partner for a small molecule such as, for example, avidin or streptavidin (which are binding partners for biotin). An antibody reagent in accordance with the principles described herein that comprises biotin linked to the hinge region of an antibody for the analyte. A chemiluminescent reagent that comprises a specific binding member for the analyte is employed as part of the detection system. The reaction medium is incubated to allow the avidin or streptavidin of the photosensitizer particles to bind to the biotin of the antibody reagent by virtue of the binding between avidin and biotin and to also allow the specific binding between the antibody of the antibody reagent in accordance with the principles described herein, which is now attached to the photosensitizer particles, to bind to the analyte of the sample and to the analyte that is part of the chemiluminescent reagent. Then, the medium is irradiated with light to excite the photosensitizer, which is capable in its excited state of activating oxygen to a singlet state. Because less of the chemiluminescent reagent is now in close proximity to the photosensitizer because of the presence of the analyte, there is less activation of the chemiluminescent reagent by the singlet oxygen and less luminescence. The medium is then examined for the presence and/or the amount of luminescence or light emitted, the presence thereof being related to the presence and/or amount of the analyte where a decrease in signal is observed in the presence of the analyte.

Another example, by way of illustration and not limitation, of an assay format for detection of vitamin D in a calibrator or in a sample is the ACMIA assay format. For the ACMIA assay format, chrome particles, which are coated with vitamin D or a vitamin D analog (chrome particle reagent), are employed as a first component. A second component is an antibody for vitamin D in accordance with the principles described herein. This antibody, pre-activated with an appropriate reactive group such as maleimide via reaction with SMCC, is linked to a reporter enzyme (for example, β-galactosidase) to form an antibody-enzyme conjugate. This conjugate is added to a reaction vessel in an excess amount, i.e., an amount greater than that required to bind all of the vitamin D analyte that might be present in a sample. A sample, which is previously subjected to treatment with a releasing agent, is treated with an antibody for vitamin D, which binds to vitamin D in the sample. The antibody-enzyme conjugate is mixed with sample in the medium to allow the vitamin D analyte to bind to the antibody. Next, the chrome particle reagent is added to bind up any excess antibody-enzyme conjugate. Then, a magnet is applied, which pulls all of the chrome particles and excess antibody-enzyme out of the suspension, and the supernatant is transferred to a final reaction container. The substrate of the reporter enzyme is added to the final reaction container, and the enzyme activity is measured spectrophotometrically as a change in absorbance over time. The amount of this signal is related to the amount of vitamin D in the sample.

Another example of an assay for vitamin D in a calibrator or in a sample is an acridinium ester label immunoassay using paramagnetic particles as a solid phase (ADVIA immunoassay). The detection system employed for this example of a vitamin D assay includes a small molecule-labeled vitamin D (capture moiety) as the small molecule conjugate or capture conjugate, binding partner for the small molecule-coated paramagnetic latex particles as a solid phase (SP), and an acridinium ester labeled antibody for vitamin D (detection antibody) in accordance with the principles described herein. The small molecule may be, for example, biotin or fluorescein and the respective binding partner may be streptavidin or antibody for fluorescein. The vitamin D may be linked to the small molecule directly or through a linking group such as, for example, a protein, e.g., bovine serum albumin (BSA). Vitamin D in a patient sample competes with vitamin D of the capture moiety for binding to the acridinium ester labeled detection anti-vitamin D antibody. The calibrator or a sample suspected of containing vitamin D is subjected to a pretreatment with 1,8-ANS. The assay may be carried out on a Centaur®, Centaur® XP or Centaur® CP apparatus (Siemens Healthcare Diagnostics Inc., Newark Del.) in accordance with the manufacturer's directions supplied therewith.

Another example of an assay for an analyte in accordance with the principles described herein is an acridinium ester label immunoassay using paramagnetic particles as a solid phase (ADVIA immunoassay). The detection system employed for this example of an assay for an analyte includes an antibody reagent in accordance with the principles described herein, in which a small molecule is linked to the hinge region of an antibody for the analyte (capture antibody) as the capture conjugate, paramagnetic latex particles as a solid phase (SP) coated with a binding partner for the small molecule of the antibody reagent, and an acridinium ester labeled analyte analog (detection hapten). The acridinium ester label may be directly bound to the analyte to form the detection hapten or a linking group may be employed including, for example, a protein such as, e.g., BSA. The analyte of a sample competes with the acridinium ester labeled detection hapten for binding with anti-analyte antibody. The sample suspected of containing the analyte may be subjected to a pretreatment with one or more of a releasing agent and a digestion agent. The assay may be carried out on a Centaur®, Centaur® XP or Centaur® CP apparatus (Siemens Healthcare Diagnostics Inc., Newark Del.) in accordance with the manufacturer's directions supplied therewith. In variations of the above acridinium ester assays, the small molecule may be, for example, biotin or fluorescein and the binding partners for the small molecule may be, for example, avidin or streptavidin or antibody for fluorescein, respectively.

The concentration of the analyte in a sample that may be assayed generally varies from about 10⁻⁵ to about 10⁻¹⁷ M, or from about 10⁻⁶ to about 10⁻¹⁴ M, for example. Considerations such as whether the assay is qualitative, semi-quantitative or quantitative (relative to the amount of the analyte present in the sample), the particular detection technique and the expected concentration of the analyte normally determine the concentrations of the various reagents.

The concentrations of the various reagents in the assay medium will generally be determined by the concentration range of interest of the analyte, the nature of the assay, and the like. However, the final concentration of each of the reagents is normally determined empirically to optimize the sensitivity of the assay over the range of interest. That is, a variation in concentration of analyte that is of significance should provide an accurately measurable signal difference. Considerations such as the nature of the signal producing system and the nature of the analytes normally determine the concentrations of the various reagents.

As mentioned above, the sample and reagents are provided in combination in the medium. While the order of addition to the medium may be varied, there will be certain preferences for some embodiments of the assay formats described herein. The simplest order of addition, of course, is to add all the materials simultaneously and determine the effect that the assay medium has on the signal as in a homogeneous assay. Alternatively, each of the reagents, or groups of reagents, can be combined sequentially. In some embodiments, an incubation step may be involved subsequent to each addition as discussed above. In heterogeneous assays, washing steps may also be employed after one or more incubation steps.

### Examination Step

In a next step of an assay method, the medium is examined for the presence of a complex comprising the analyte and antibody for the presence and/or amount of the complex indicates the presence and/or amount of the analyte in the sample.

The phrase “measuring the amount of an analyte” refers to the quantitative, semiquantitative and qualitative determination of the analyte. Methods that are quantitative, semiquantitative and qualitative, as well as all other methods for determining the analyte, are considered to be methods of measuring the amount of the analyte. For example, a method, which merely detects the presence or absence of the analyte in a sample suspected of containing the analyte, is considered to be included within the scope of the present invention. The terms “detecting” and “determining,” as well as other common synonyms for measuring, are contemplated within the scope of the present invention.

In many embodiments the examination of the medium involves detection of a signal from the medium. The presence and/or amount of the signal is related to the presence and/or amount of the analyte in the sample. The particular mode of detection depends on the nature of the signal producing system. As discussed above, there are numerous methods by which a label of a signal producing signal can produce a signal detectable by external means. Activation of a signal producing system depends on the nature of the signal producing system members.

Temperatures during measurements generally range from about 10° C. to about 70° C. or from about 20° C. to about 45° C., or about 20° C. to about 25° C., for example. As mentioned above, calibrators are formed using known concentrations of the analyte. In one approach standard curves are formed using known concentrations of the analyte or calibrators. In accordance with the principles described herein, signal ratio between calibrators in an assay for an analyte is enhanced over calibrators that are assayed wherein a moiety is conjugated to an antibody at other than the hinge region of the antibody.

Luminescence or light produced from any label can be measured visually, photographically, actinometrically, spectrophotometrically, such as by using a photomultiplier or a photodiode, or by any other convenient means to determine the amount thereof, which is related to the amount of analyte in the medium. The examination for presence and/or amount of the signal also includes the detection of the signal, which is generally merely a step in which the signal is read. The signal is normally read using an instrument, the nature of which depends on the nature of the signal. The instrument may be, but is not limited to, a spectrophotometer, fluorometer, absorption spectrometer, luminometer, and chemiluminometer, for example.

### Kits Comprising Reagents for Conducting Assays

An antibody reagent in accordance with the principles described herein and other reagents for conducting a particular assay for an analyte may be present in a kit useful for conveniently performing an assay for the determination of the analyte. In some embodiments a kit comprises in packaged combination a biotin-binding partner such as, for example, avidin or streptavidin, associated with a particle, biotinylated antibody in accordance with the principles described herein and a labeled analyte analog. The kit may further include other reagents for performing the assay, the nature of which depend upon the particular assay format.

The reagents may each be in separate containers or various reagents can be combined in one or more containers depending on the cross-reactivity and stability of the reagents. The kit can further include other separately packaged reagents for conducting an assay such as additional specific binding pair members, signal producing system members, and ancillary reagents, for example.

The relative amounts of the various reagents in the kits can be varied widely to provide for concentrations of the reagents that substantially optimize the reactions that need to occur during the present methods and further to optimize substantially the sensitivity of an assay. Under appropriate circumstances one or more of the reagents in the kit can be provided as a dry powder, usually lyophilized, including excipients, which on dissolution will provide for a reagent solution having the appropriate concentrations for performing a method or assay using a compound reagent in accordance with the principles described herein. The kit can further include a written description of a method utilizing reagents that include a compound reagent in accordance with the principles described herein.

The phrase “at least” as used herein means that the number of specified items may be equal to or greater than the number recited. The phrase “about” as used herein means that the number recited may differ by plus or minus 10%; for example, “about 5” means a range of 4.5 to 5.5.

The following discussion is directed to specific examples in accordance with the principles described herein by way of illustration and not limitation; the specific examples are not intended to limit the scope of the present disclosure and the appended claims. Numerous modifications and alternative compositions, methods, and systems may be devised without departing from the spirit and scope of the present disclosure.

### EXAMPLES

Unless otherwise indicated, materials in the experiments below may be purchased from the Sigma-Aldrich Chemical Corporation (St. Louis Mo.). Parts and percentages disclosed herein are by weight to volume unless otherwise indicated.

### DEFINITIONS

mg=milligram

g=gram(s)

ng=nanogram(s)

mL=milliliter(s)

μL=microliter(s)

μmol=micromolar

° C.=degrees Centigrade

min=minute(s)

sec=second(s)

hr=hour(s)

w/v=weight to volume

v/v=volume to volume

TLC=thin layer chromatography

HPLC=high performance liquid chromatography

EDTA=ethylenediaminetetraacetate

PEG=polyethylene glycol

EtOAc=ethyl acetate

DMF=dimethylformamide

DMSO=dimethylsulfoxide

MeOP=1-methoxy-2-propanol

MES=2-(N-morpholino)ethanesulfonic acid

DI water=distilled water

UPA=Ultra Particle Analyzer

LOCI=luminescent oxygen channeling immunoassay

EDA=Ethylenediamine

EDAC=N-(3-Dimethylaminopropyl)-N′-ethylcarbodiimide hydrochloride

sulfoNHS=sulfo N-hydroxysuccinimide

### Preparation of Antibody Biotinylated Through Hinge Region

A solution (1.4 mL at 2.8 mg/mL) of vitamin D antibody (sheep monoclonal from Bioventix, Farnham, Surrey, UK) was mixed with 0.155 mL of a freshly prepared solution of DTT (15.4 mg/mL in 100 mM phosphate, 50 mM EDTA, pH 6.0 buffer). Protein solution was heated for 1 hour in a water bath at 37° C. Protein mixture was passed through a SEPHADEX® G25 column (1.6×30 cm) prepared and eluted with a solution containing 0.1 M phosphate, 5 mM EDTA, 0.1% Tween 20, pH 7.60. Protein-containing fractions (as determined by measuring absorption at 280 nm) obtained from SEPHADEX® G25 were pooled.

Titration of free sulfhydryls generated in the antibody during the above reduction step was determined by adding 20 μL of a solution of ALDRITHIOL™ 4 (Sigma-Aldrich Chemical Corporation, part number 143057) to 0.4 mL of the antibody solution containing 0.27 mg/mL antibody. A change in absorption at 324 nm (molar extinction coefficient of 19800 M⁻¹cm⁻¹) was used to calculate the number of free sulfhydryls. This titration showed 13.1 free sulfhydryls/mole of antibody.

The remaining antibody pool was concentrated in an AMICON® (YM10) device to 1.84 mL solution containing 1.9 mg/mL antibody. This vortex mixed antibody solution was slowly mixed with 63 μL of a freshly prepared aqueous solution (2 mg/mL) of iodoacetyl-PEG₂-biotin (Pierce/Thermo Scientific, Rockford Ill., part number 21334). The amount of iodoacetyl-PEG₂-biotin added represents a 10-fold molar challenge of the biotinylating agent with the antibody. The antibody mixture was shaken for 3 hr at 25° C. in an incubator. Excess free sulfhydryls were quenched by addition of 36 μL of a fresh aqueous solution of N-ethylmaleimide (20 mg/mL).

After additional 30 min at room temperature, clear antibody solution was purified by passage through a 1.6×30 cm SEPHADEX® G25 column prepared and eluted with a solution of 10 mM PO₄, 300 mM NaCl, pH 7.0 containing 0.1% TWEEN® 20. Antibody-containing fractions, as determined by absorption at 280 nm, were pooled and concentrated in an AMICON® (YM10) to 2.8 mL. This pool was shown by absorption at 280 nm to contain 1.21 mg/mL protein. The product was preserved with neomycin sulfate to 0.1 mg/mL and PROCLIN® 300 (to 0.15% v/v) and then filtered using a 0.2 μm ACRODISC® syringe filter (Pall Corporation, Andheri (East), Mumbai).

### Preparation of Antibody Biotinylated not Through Hinge Region

A solution (0.8 mL at 2.63 mg/mL) of vitamin D antibody (sheep monoclonal from Bioventix) in 10 mM PO₄, 300 mM NaCl, pH 7.0 was mixed with 43.2 μL of an aqueous solution (2.0 mg/mL) of NHS-dPEG®4-biotin (Quanta Biodesign Ltd., Powell Ohio, part number 10200). The amount of biotinylation reagent added represents a 10-fold molar challenge of the biotinylating agent with the antibody. The reaction mixture was incubated at room temperature for 3 hr and then the reaction was quenched by addition of 80 μL of 0.5 M TRIS. The reaction mixture was subjected to buffer exchange with 10 mM PO₄, 300 mM NaCl, pH 7.0 in an AMICON® (YM10) device until absorption at 260 nm of the effluent was ≦0.03. The antibody solution (1.04 mL at 2.1 mg/mL protein) was mixed with 10 μL of PROCLIN® 300 and 10 μL of an aqueous solution of neomycin sulfate (10 mg/mL) filtered using a 0.2 um ACRODISC® syringe filter (Pall Corporation) and was stored at 2-8° C.

### Preparation of EPRM-EDA Beads

EPRM beads (2000 mg, 20.0 mL) are added to a 40-mL vial. The EPRM beads are prepared by a procedure similar to that described in U.S. Pat. No. 7,179,660 and the chemiluminescent compound is 2-(4-(N,N, di-tetradecyl)-anilino-3-phenyl thioxene with europium chelate. EDA (800 mg, 890 μL) is combined with 10 mL MES pH 6 buffer (the “Buffer”) and about 4.2 mL 6N HCl. The pH of the mixture is, or is adjusted to be, about 6.9. The EDA solution is added to the EPRM beads with vortexing and the mixture is rocked at room temperature for 15 minutes. Sodium cyanoborohydride (400 mg) is combined in a 15 mL vial with 10 mL DI water and the combination is added to the bead mixture from above. The mixture is shaken at 37° C. for 18-20 hours. The beads are transferred to six 40 mL centrifuge tubes. MES buffer is added to bring the volume to 35 mL and the mixture is centrifuged at 19,000 rpm for 30 min. The supernatant is decanted and the beads are re-suspended in 2 mL of the Buffer with a stir-rod and additional Buffer is added to 35 mL. The mixture is sonicated at 18 Watts power for 30 sec, using ice to keep the mixture cold. The wash/sonication step is performed 4 times to remove all excess unreacted EDA still present in the reaction mixture. After the last MES Buffer centrifugation, 2 mL of the Buffer containing 5% MeOP and 0.1% Tween® 20 (the “second Buffer”) is added to the tubes for the re-suspension step. Additional second buffer is added to 35 mL before sonication. The bead suspension is centrifuged at 19,000 rpm for 30 min. The supernatant is discarded. The final sonication used 12 mL of the second Buffer in each tube to give a 25 mg/mL dilution. Particle size is 277 nm as determined on a UPA instrument.

The EPRM chemibead is prepared in a manner similar to the method described in U.S. Pat. No. 6,153,442 and U.S. Patent Application Publication No. 20050118727A, the relevant disclosures of which are incorporated herein by reference. The EPRM chemibead comprises an aminodextran inner layer and a dextran aldehyde outer layer having free aldehyde functionalities. See, for example, U.S. Pat. Nos. 5,929,049, 7,179,660 and 7,172,906, the relevant disclosures of which are incorporated herein by reference. The reaction is carried out at a temperature of about 4° C. to about 40° C. for a period of about 16 to about 64 hours at a pH of about 5.5 to about 7.0, or about 6, in a buffered aqueous medium employing a suitable buffer such as, for example, MES. The reaction is quenched by addition of a suitable quenching agent such as, for example, carboxymethoxyamine hemihydrochloride (CMO), and subsequent washing of the particles.

Aldehyde groups on the outer dextran aldehyde layer are reacted with ethylene diamine under reductive amination conditions to form reagent EPRM-EDA having pendant moieties comprising an ethylene chain and a terminal amine group. The reductive amination conditions include the use of a reducing agent such as, for example, a metal hydride. The reaction is carried out in an aqueous medium at a temperature during the reaction of about 20° C. to about 100° C. for a period of about 1 hour to about 48 hours.

Synthesis of 25-OH Vitamin D₃ 3-carbamate (25-OH Vitamin D₂ 3-carbamate)

A mixture of 22 mg (55 μmol) 25-OH VD₃ purchased from ChemReagents.com, Sugarland Tex., 100 mg (420 μmol) disuccinimidyl carbonate (DSC), 100 μL triethylamine in 1 mL anhydrous acetonitrile in a 5-ml flask (covered with foil) was stirred at room temperature for 18 hr under nitrogen to prepare activated 25-OH VD₃. TLC (EtOAc:Hexane=2:1) showed no starting material left. A suspension was prepared by adding 150 mg of carboxymethoxylamine hemihydrochloride (CMO), 0.3 ml triethylamine and 1 ml DMF to a 10 ml flask. A solution containing activated 25-OH VD₃ was added dropwise to the CMO suspension with stirring, which was continued for another 18 hr. Vacuum was applied to remove the solvents as much as possible (the heating bath temperature should not be over 50° C.). EtOAc (25 ml) was added to the residue, which was washed three times with 2 ml brine. The organic phase was dried with anhydrous Na₂SO₄ and was filtered; solvent was removed using rotary evaporator. Crude product (42 mg), obtained after drying, was purified by HPLC. Pure product (24 mg) was obtained after being dried under high vacuum. The product was dissolved into 1.2 ml anhydrous DMSO to prepare a 20 mg/mL solution. Aliquots were transferred into vials, which were kept at −70° C.

### Coupling of EPRM-EDA and 25-OH Vitamin D₃ 3-Carbamate to Give Chemibead Reagent

25-OH Vitamin D₃ 3-Carbamate (10 μL of aliquot in DMSO prepared as described above) (0.2 mg) was added to a 2-mL vial. EDAC (6.8 mg) and sulfoNHS (9.4 mg) plus 2.27 mL dry DMSO (3 mg/mL) were added to a 5-mL vial. The EDAC/sulfoNHS solution (190 μL) was combined with the contents of the 2-mL vial from above (1 mg/mL) to prepare activated 25-OH vitamin D₃ 3-carbamate. The mixture was allowed to rotate at room temperature for 18 hr.

A 0.4 mL aliquot of a 16% GAFAC® surfactant solution (GAF Corporation, Wayne N.J.) (0.15%) was diluted to 1.6% with 3.6 mL DI water.

To a 10-mL round bottom flask equipped with a stir-bar was added 2.0 mL (200 mg) EPRM-EDA (prepared as described above) followed by 260 μL 1.6% GAFAC® surfactant solution (0.15%) with moderate stirring. To a small test tube was added 504 μL anhydrous DMSO followed by 60 μL (0.06 mg) activated Vitamin D₃-3-carbamate prepared as described above; and the mixture was added to the EPRM-EDA bead mixture. The total DMSO content of the bead suspension was 20%. The reaction vessel was allowed to stir overnight at room temperature. Then, the beads were washed by means of diafiltration.

The reaction mixture containing beads was diluted to 20 mL by addition of 10% MeOP/1% GAFAC®/MES pH6 buffer. The mixture was diafiltered with 5 volumes of the MOP-GAFAC-MES pH6 buffer and then sonicated with a probe sonicator at 18-21 Watts using ice to keep the mixture cold. The diafiltration/sonication continued through 50 volumes with effluent samples being taken at 35, 40, 45 and 50 volumes. The buffer was changed to LOCI Hapten Wash Buffer (50 mM HEPES, 300 mM NaCl, 1 mM EDTA, 0.01% neomycin sulfate, 0.1% TRITON® 405X and 0.15% PROCLIN® 300, pH 7.2) with 10 volumes being used. The mixture was reduced to about 7 mL and a particle size analysis by UPA showed to be 289 nm. Percent solids were determined and the bead lot was brought up to 10 mg/mL with LOCI Hapten Wash Buffer pH7.2. Yield was 160.4 mg.

### Assay for Vitamin D Analyte

Assays were carried out on a DIMENSION® VISTA® analyzer (Siemens Healthcare Diagnostics Inc., Deerfield, Ill.) following the protocol for a LOCI assay and using calibrator solutions containing varying amounts of 25-hydroxyvitamin D₃. In this example, the assay uses, as a chemiluminescent reagent, the chemibead reagent prepared as described above. Calibrator samples are reacted first with the biotinylated antibody reagent prepared as described above and then with the chemibead reagent. The chemibeads bind to the fraction of the monoclonal antibody binding sites that is not occupied by analyte from the calibrator sample. Subsequently, streptavidin coupled sensitizer beads are added to the reaction mixture. This leads to the formation of chemibead/sensibead pairs whose concentration is inversely related to the concentration of 25-hydroxyvitamin D₃. Upon illumination at 680 nm, the sensitizer beads generate singlet oxygen which diffuses into the chemibeads which are paired with sensibeads, reacts with the olefinic dye and triggers a chemiluminescent signal at approximately 612 nm which is inversely related to the analyte concentration.

The streptavidin-sensitizer bead (“sensibead(s)”) is prepared using a method analogous to that described in U.S. Pat. Nos. 6,153,442, 7,022,529, 7,229,842 and U.S. Patent Application Publication No. 20050118727A. The photosensitizer was bis-(trihexyl)-silicon-t-butyl-phthalocyanine. The concentration of sensibead reagent was 200 μg/mL in HEPES buffer, pH 8.0 containing 150 mM NaCl. The EPRM-EDA-25-OH Vitamin D₃ particle reagent prepared as described above was employed as the “chemibead reagent” at a concentration of 200 μg/mL in HEPES buffer, pH 7.2, containing 150 mM NaCl and 0.1% detergent.

At time t=zero sec, 20 μL biotinylated antibody reagent and 20 μL water were added to a reaction vessel. Sample, 12 μL, was added 21.6 seconds later, followed by 8 μL water. At t=414.0 seconds, 40 μL chemibead reagent was added followed by 20 mL of water. Sensibead reagent was then dispensed at 457.2 seconds. Measurements were taken 601.2 seconds after initiation of the reaction sequence.

Calibrator assays were also carried out using the antibody biotinylated not through hinge region prepared as described above.

The results are summarized in Table 1 below.

TABLE 1

kcounts

| | | |
|---|---|---|
| Calibrator | Biotinylation in | Biotinylation not in |
| Vitamin D (ng/mL) | hinge region | hinge region |

| | | | |
|---|---|---|---|
| L1 | 0 | 3551 | 1155 |
| L2 | 5 | 3256 | 1205 |
| L3 | 23 | 2339 | 1007 |
| L4 | 216 | 347 | 535 |
| L5 | 528 | 116 | 272 |

As can be seen from Table 1, biotinylation of the vitamin D₃ antibody through the hinge region significantly improved the assay kcounts (3 times) and signal separation (more than 4 times) in comparison to the biotinylation not through the hinge region of the antibody. For example, the ratio L1/L4 is 10.23 for biotinylation of the antibody through the hinge region whereas the ratio L1/L4 for biotinylation of the antibody through other than the hinge region is 2.16. Separation is a major factor influencing precision and sensitivity of competitive immunoassays.

All publications and patent applications cited in this specification are herein incorporated by reference as if each individual publication or patent application were specifically and individually indicated to be incorporated by reference.

It should be understood that the above-described examples are merely illustrative of some of the many specific examples that represent the principles described herein. Clearly, those skilled in the art can readily devise numerous other arrangements without departing from the scope as defined by the following claims.

What is claimed is:

1. A method of enhancing signal ratio between calibrators in an assay for an analyte, the method comprising:
conducting the assay for the analyte with zero concentration of analyte in a first calibrator to determine a first signal level, wherein reagents employed in the assay comprise an antibody reagent comprising an antibody for the analyte wherein a hinge region of the antibody is conjugated to a moiety;
conducting the assay for the analyte with a second concentration of analyte in a second calibrator to determine a second signal level wherein the second analyte concentration is greater than zero and wherein the reagents employed in the assay comprise the antibody reagent; and
determining a ratio of the first signal level to the second signal level.

2. The method of claim 1 wherein the moiety is selected from the group consisting of a member of a signal producing system or a member of a specific binding pair.

3. The method according to claim 1 wherein the assay is a homogeneous assay.

4. The method according to claim 1 wherein the assay is a heterogeneous assay.

5. The method according to claim 1 wherein the assay is a competitive assay.

6. The method according to claim 1 wherein the assay is a non-competitive assay.

7. The method according to claim 1 wherein the reagents further comprise an analog of the analyte wherein the analog comprises a label.

8. The method according to claim 1 wherein the antibody for the analyte is an antibody from a sheep source.

9. The method according to claim 8 wherein at least the antibody for the analyte or the second binding partner comprises a label.

10. The method according to claim 1 wherein the moiety is a member of a signal producing system.

11. The method according to claim 9 wherein the member of a signal producing system is a label.

12. The method according to claim 1 wherein the reagents further comprise a particle.

13. The method according to claim 1 wherein the reagents further comprise a photosensitizer reagent and a chemiluminescent particle.

14. The method according to claim 13 wherein the photosensitizer reagent comprises a particle.

15. The method according to claim 1 wherein the moiety is conjugated to the hinge region of the antibody by means of a thioether linkage.

16. A method of enhancing signal ratio between calibrators in an assay for an analyte, the method comprising:
conducting the assay for the analyte with zero concentration of analyte in a first calibrator to determine a first signal level, wherein reagents employed in the assay comprise (i) an antibody reagent comprising an antibody having a thioether linkage between a hinge region of the antibody and a small molecule, (ii) a chemiluminescent particle reagent comprising an analyte analog, and (iii) a photosensitizer particle reagent comprising a small molecule-binding moiety;
conducting the assay for the analyte with a second concentration of analyte in a second calibrator to determine a second signal level wherein the second concentration of analyte is greater than zero and wherein the reagents employed in the assay comprise the antibody reagent, the chemiluminescent particle reagent and the photosensitizer particle reagent; and
determining the ratio of the first signal level to the second signal level.

17. The method according to claim 16 wherein the assay is conducted for the analyte with a third concentration of analyte in a third calibrator to determine a third signal level wherein the third concentration of analyte is greater than zero and less than the second concentration of analyte in the second calibrator.

18. The method according to claim 17 wherein the assay is conducted for the analyte with a fourth concentration of analyte in a fourth calibrator to determine a fourth signal level wherein the fourth concentration of analyte is greater than zero and greater than the second concentration of analyte in the second calibrator.

19. The method according to claim 18 wherein the assay is conducted for the analyte with a fifth concentration of analyte in a fifth calibrator to determine a fifth signal level wherein the fifth concentration of analyte is greater than zero and greater than the second concentration of analyte in the second calibrator.

20. The method according to claim 15 wherein the small molecule is selected from the group consisting of biotin, fluorescein, rhodamine and nitro- and carboxy-derivatives of benzene.
